(19) United States
(12) Patent Application Publication
(10) Pub. No.: US 2026/0325596 A1
(43) Pub. Date: Oct. 22, 2026

(54) PAIN RELIEF FORMULATION, PREPARATION METHOD THEREFOR, AND APPLICATION THEREOF

(71) Applicant: TONGJI HOSPITAL TONGJI MEDICAL COLLEGE OF HUST, Wuhan (CN)

(72) Inventors: Xueqin CAO, Wuhan (CN); Ningbo LI, Wuhan (CN); Jiaoli SUN, Wuhan (CN); Baowen LIU, Wuhan (CN); Bo JIAO, Wuhan (CN)

(21) Appl. No.: 19/378,340

(22) Filed: Nov. 4, 2025

(30) Foreign Application Priority Data
Apr. 17, 2025 (CN) 202510480045.1

(51) Int. Cl. A61K 36/233; A61K 31/045; A61K 47/40; A61P 29/00

(52) U.S. Cl. CPC A61K 36/233; A61K 31/045; A61K 47/40; A61P 29/00; A61K 2236/15; A61K 2236/33; A61K 2236/51; A61K 2236/53

(57) ABSTRACT

A pain relief formulation, a preparation method therefor, and an application thereof are provided, relating to the technical field of pain relief formulations. The pain relief formulation includes dexborneol, a propylene glycol extract of Bupleurum, and a pharmaceutically acceptable excipient. A mass ratio of the dexborneol to the propylene glycol extract of Bupleurum is (1-2.5):1. The propylene glycol extract of Bupleurum is prepared through the following method: grinding Bupleurum into Bupleurum powder, mixing the Bupleurum powder with propylene glycol, followed by ultrasonic extracting and filtering to obtain a filtrate, collecting the filtrate, and drying the filtrate to obtain the propylene glycol extract of the Bupleurum. The pain relief formulation is applicable in relieving acute pain and postherpetic neuralgia, thereby expanding types of available pain relief formulations.

### CROSS-REFERENCE TO RELATED APPLICATION

This application claims priority to Chinese Patent Application No. 202510480045.1, filed on Apr. 17, 2025, which is herein incorporated by reference in its entirety.

### TECHNICAL FIELD

The disclosure relates to the technical field of pain relief formulations, and more particularly to a pain relief formulation, a preparation method therefor, and an application thereof.

### BACKGROUND

Many diseases are accompanied by pain symptoms, such as those caused by bacterial and viral infections. Pain affects normal life of a patient, so relieving pain is necessary.

In related art, means for relieving pain mainly include taking ibuprofen, acetaminophen, and other formulations, using a lidocaine patch for local anesthesia, pulsed radio frequency therapy, and short-term spinal cord electrical stimulation. Among them, the means for relieving pain through formulations are more convenient and widely used. For example, formulations in the art that can be used to relieve pain includes: (1) antiviral formulations, such as acyclovir tablets and valaciclovir tablets, which help kill viruses in body and relieve pain symptoms; (2) non-steroidal anti-inflammatory formulations, such as ibuprofen tablets, indomethacin tablets, and diclofenac sodium sustained-release capsules, which help relieve fever and pain; (3) central nervous system formulations, such as gabapentin capsules, pregabalin capsules, etc., which mildly inhibit a central nervous system and relieve discomfort symptoms such as itching; (4) tricyclic antidepressants: such as amitriptyline hydrochloride tablets and imipramine hydrochloride tablets, etc., which can be used for treatment in a case where patients with postherpetic neuralgia suffer from anxiety, depression and other symptoms due to long-term pain and even generate a cognition of aggravated pain; and (5) traditional Chinese medicine (TCM) formulations for promoting blood circulation and regulating qi, such as Chaihu Shugan powder (also referred to as Bupleurum liver-regulating powder), Taohong Siwu decoction (also referred to as persica and Carthamus four substances decoction), and Xuefu Zhuyu decoction (also referred to as decoction for removing stasis from the blood mansion), which can help promote blood circulation and resolve blood stasis, thereby relieving the pain symptoms. However, due to differences in physical constitution among patients, sensitivity and resistance of different patients to analgesic medicines in the art vary, so it is necessary to continue developing pain relief formulations.

### SUMMARY

To solve the aforementioned technical problems, the disclosure provides a pain relief formulation, a preparation method therefor, and an application thereof. The pain relief formulation is applicable in relieving acute pain and postherpetic neuralgia.

The disclosure aims to provide the pain relief formulation, including dexborneol, a propylene glycol extract of Bupleurum, and a pharmaceutically acceptable excipient. A mass ratio of the dexborneol to the propylene glycol extract of Bupleurum is (1-2.5):1. The propylene glycol extract of Bupleurum is prepared through the following method: grinding Bupleurum into Bupleurum powder, mixing the Bupleurum powder with propylene glycol, followed by ultrasonic extracting and filtering to obtain a filtrate, collecting the filtrate, and drying the filtrate to obtain the propylene glycol extract of the Bupleurum.

According to research results of the disclosure, in relieving the acute pain and the postherpetic neuralgia, the dexborneol and the propylene glycol extract of Bupleurum in a mixture formulation composed of a combination of the dexborneol and the propylene glycol extract of Bupleurum and the pharmaceutically acceptable excipient have a synergistic analgesic effect. In addition, in the pain relief formulation provided by the disclosure, the dexborneol is mainly used to relieve pain associated with nerve injury, which can regulate an immune function of a body and inhibit pain conduction, and the propylene glycol extract of Bupleurum contains saikosaponin, volatile oils, and sterols, which can promote blood circulation and reduce conduction of pain signals. Therefore, after combining the dexborneol with the propylene glycol extract of Bupleurum, effect of reducing the conduction of pain signals is enhanced, the acute pain can be relieved, and the postherpetic neuralgia is also significantly relieved.

In addition, according to the research results of the disclosure, the dexborneol alone exhibits a weak analgesic effect in relieving the acute pain or the postherpetic neuralgia; and the propylene glycol extract of Bupleurum alone also exhibits a weak analgesic effect in relieving the acute pain or the postherpetic neuralgia.

In addition, after the Bupleurum is ground into the Bupleurum powder, a particle size decreases and a surface area increases, allowing for more sufficient contact with a solvent of the propylene glycol. With assistance of ultrasonic extraction, the saikosaponin, the volatile oils, and the sterols can dissolve in the propylene glycol. Subsequently, after filtering to obtain the filtrate, collecting the filtrate, and drying the filtrate, the propylene glycol extract of Bupleurum rich in the saikosaponin, volatile oils, and sterols can be obtained.

In an embodiment, the pain relief formulation exhibits a better pain-relieving effect when a mass ratio of the dexborneol to the propylene glycol extract of Bupleurum is 2:1.

In an embodiment, conditions for the ultrasonic extracting include a power of 400 watts (W) and a duration of 30 minutes (min). Under these conditions for the ultrasonic extracting, the saikosaponin, the volatile oils, and the sterols have high solubility in the propylene glycol.

In an embodiment, a preparation method for the propylene glycol extract of Bupleurum is specifically as follows: grinding the Bupleurum into the Bupleurum powder with a particle size of 40 meshes, mixing the Bupleurum powder with the propylene glycol according to a ratio of 1 kilogram (kg) to 8 liters (L), followed by ultrasonic extracting for 30 min under the power of 400 W, and filtering with a 50-mesh screen to obtain the filtrate, collecting the filtrate, and drying the filtrate to obtain the propylene glycol extract of Bupleurum.

In an embodiment, the pharmaceutically acceptable excipient is a combination of the propylene glycol and water, the propylene glycol, or a combination of hydroxypropyl-β-cyclodextrin, the propylene glycol, and the water.

It should be noted that a term “combination” in the pharmaceutically acceptable excipient refers to use of multiple raw materials of the pharmaceutically acceptable excipient. The multiple raw materials can be mixed before use or used separately.

In an embodiment, a concentration of the dexborneol in the pain relief formulation is in a range of 60 nanograms per milliliter (ng/mL) to 80 ng/mL.

In an embodiment, the concentration of the dexborneol in the pain relief formulation is 75 ng/mL.

The preparation method for the pain relief formulation provided by the disclosure includes the following steps: mixing the dexborneol, the propylene glycol extract of Bupleurum, and the pharmaceutically acceptable excipient to obtain a mixture. The mixture is the pain relief formulation.

In an embodiment, when the pharmaceutically acceptable excipient is the combination of the propylene glycol and the water, the preparation method for the pain relief formulation includes the following steps: dissolving the dexborneol in the propylene glycol to obtain a first dexborneol liquid; dissolving the propylene glycol extract of Bupleurum in a water-propylene glycol mixture to obtain a first propylene glycol extract liquid of Bupleurum; and mixing the first dexborneol liquid with the first propylene glycol extract liquid of Bupleurum to thereby obtain a first pain relief formulation suitable for intramuscular injection.

In an embodiment, when the pharmaceutically acceptable excipient is the propylene glycol, the preparation method for the pain relief formulation includes the following steps: dissolving the dexborneol in the propylene glycol to obtain the first dexborneol liquid; dissolving the propylene glycol extract of Bupleurum in the propylene glycol to obtain a second propylene glycol extract liquid of Bupleurum; and mixing the first dexborneol liquid with the second propylene glycol extract liquid of Bupleurum to thereby obtain a second pain relief formulation suitable for nasal drops.

In an embodiment, when the pharmaceutically acceptable excipient is the combination of the hydroxypropyl-β-cyclodextrin, the propylene glycol, and the water, the preparation method for the pain relief formulation includes the following steps: encapsulating the dexborneol in an excipient of the hydroxypropyl-β-cyclodextrin to obtain a second dexborneol liquid; dissolving the propylene glycol extract of Bupleurum in the water-propylene glycol mixture to obtain the first propylene glycol extract liquid of Bupleurum; and mixing the second dexborneol liquid with the first propylene glycol extract liquid of Bupleurum to thereby obtain a third pain relief formulation suitable for intramuscular injection.

The application of the pain relief formulation provided by the disclosure refers to relieving the acute pain or relieving the postherpetic neuralgia.

In an embodiment, in the application of the pain relief formulation, the postherpetic neuralgia refers to pain caused by varicella-zoster virus.

Compared with the related art, the disclosure has the following beneficial effects.

The pain relief formulation provided by the disclosure is composed of the dexborneol, the propylene glycol extract of Bupleurum, and the pharmaceutically acceptable excipient. The mass ratio of the dexborneol to the propylene glycol extract of Bupleurum is (1-2.5):1. The propylene glycol extract of Bupleurum is prepared through the following method: grinding the Bupleurum into the Bupleurum powder, mixing the Bupleurum powder with the propylene glycol, followed by ultrasonic extracting and filtering to obtain the filtrate, collecting the filtrate, and drying the filtrate to obtain the propylene glycol extract of the Bupleurum. The combination of the dexborneol and the propylene glycol extract of Bupleurum is mixed with the pharmaceutically acceptable excipient to obtain the mixture formulation. The dexborneol and the propylene glycol extract of Bupleurum in the pain relief formulation provided by the disclosure have the synergistic analgesic effect in relieving the acute pain and the postherpetic neuralgia.

### DETAILED DESCRIPTION OF EMBODIMENTS

To enable those skilled in the art to better understand and implement technical solutions of the disclosure, the disclosure will be further described with reference to specific embodiments.

In description of the disclosure, unless otherwise indicated, all reagents used are commercially available, and all methods used are conventional technologies in the art.

In the following embodiments and experiments, dexborneol is purchased from Jiangxi Linke Longnao Technology Co., Ltd. and made by extracting and processing fresh branches and leaves of Cinnamomum camphora. A content of the dexborneol is larger than or equal to 96%. A packaging specification is 500 grams (g) per bag. According to medical research results, the dexborneol has analgesic, anti-inflammatory, and sleep improving effects.

Bupleurum is purchased from a traditional Chinese medicine herb store. According to the medical research results, the Bupleurum has effects of antipyretic and anti-inflammatory, soothing liver and relieving depression, elevating yang qi, and harmonizing exterior and interior.

In a pain relief formulation provided by the disclosure, substances that can relieve pain are the dexborneol and propylene glycol extract of Bupleurum. When combined with a pharmaceutically acceptable excipient, the dexborneol and the propylene glycol extract of Bupleurum can be formulated into intramuscular injections or nasal drops, both of which exhibit efficacy in relieving pain. The relieving pain refers to relieving acute pain or relieving postherpetic neuralgia.

When the pharmaceutically acceptable excipient is a combination of propylene glycol and water, a preparation method for the pain relief formulation includes the following steps. The dexborneol is dissolved in the propylene glycol to obtain a first dexborneol liquid. The propylene glycol extract of Bupleurum is dissolved in a water-propylene glycol mixture to obtain a first propylene glycol extract liquid of Bupleurum. The first dexborneol liquid is mixed with the first propylene glycol extract liquid of Bupleurum to thereby obtain a first pain relief formulation suitable for intramuscular injection. A volume ratio of the water to the propylene glycol in the water-propylene glycol mixture is 10:1.

When the pharmaceutically acceptable excipient is the propylene glycol, the preparation method for the pain relief formulation includes the following steps. The dexborneol is dissolved in the propylene glycol to obtain the first dexborneol liquid. The propylene glycol extract of Bupleurum is dissolved in the propylene glycol to obtain a second propylene glycol extract liquid of Bupleurum. The first dexborneol liquid is mixed with the second propylene glycol extract liquid of Bupleurum to thereby obtain a second pain relief formulation suitable for nasal drops.

When the pharmaceutically acceptable excipient is a combination of hydroxypropyl-β-cyclodextrin, the propylene glycol, and the water, the preparation method for the pain relief formulation includes the following steps. The dexborneol is encapsulated in an excipient of the hydroxypropyl-β-cyclodextrin to obtain a second dexborneol liquid. The propylene glycol extract of Bupleurum is dissolved in the water-propylene glycol mixture to obtain the first propylene glycol extract liquid of Bupleurum. The second dexborneol liquid is mixed with the first propylene glycol extract liquid of Bupleurum to thereby obtain a third pain relief formulation suitable for intramuscular injection. The volume ratio of the water to the propylene glycol in the water-propylene glycol mixture is 10:1.

Embodiments of the disclosure and corresponding pain-relieving effects will be illustrated as follows with reference to the pain relief formulations with a dosage form of intramuscular injection and related animal experiments.

### Embodiment 1

A pain relief formulation is composed of dexborneol, propylene glycol extract of Bupleurum, and a pharmaceutically acceptable excipient. A mass ratio of the dexborneol to the propylene glycol extract of Bupleurum is 1:1.

A preparation method of the propylene glycol extract of Bupleurum is specifically as follows. Bupleurum is ground into Bupleurum powder with a particle size of 40 meshes. The Bupleurum powder is mixed with propylene glycol according to a ratio of 1 kg:8 L, then ultrasonic extracted for 30 min under a power of 400 W, and filtered with a 50-mesh screen, to thereby obtain a filtrate. The filtrate is collected and then freeze-dried at −20 degrees Celsius (° C.) to thereby obtain the propylene glycol extract of Bupleurum.

The pharmaceutically acceptable excipient is a combination of the propylene glycol and water.

A preparation method for the pain relief formulation is specifically as follows. The dexborneol is dissolved in the propylene glycol to obtain a dexborneol liquid. The propylene glycol extract of Bupleurum is dissolved in a water-propylene glycol mixture to obtain a propylene glycol extract liquid of Bupleurum. A volume ratio of the water to the propylene glycol in the water-propylene glycol mixture is 10:1. According to a mass ratio of the dexborneol to the propylene glycol extract of Bupleurum of 1:1, the dexborneol liquid and the propylene glycol extract liquid of Bupleurum are prepared, and then mixed to thereby obtain the pain relief formulation. A concentration of the dexborneol in the pain relief formulation is 75 ng/mL.

### Embodiment 2

A pain relief formulation is composed of dexborneol, propylene glycol extract of Bupleurum, and a pharmaceutically acceptable excipient. A mass ratio of the dexborneol to the propylene glycol extract of Bupleurum is 1.5:1.

A preparation method for the propylene glycol extract of Bupleurum is specifically as follows. Bupleurum is ground into Bupleurum powder with a particle size of 40 meshes. The Bupleurum powder is mixed with propylene glycol according to a ratio of 1 kg:8 L, then ultrasonic extracted for 30 min under a power of 400 W, and filtered with a 50-mesh screen, to thereby obtain a filtrate. The filtrate is collected and then freeze-dried at −20° C. to thereby obtain the propylene glycol extract of Bupleurum.

The pharmaceutically acceptable excipient is a combination of the propylene glycol and water.

A preparation method for the pain relief formulation is specifically as follows. The dexborneol is dissolved in the propylene glycol to obtain a dexborneol liquid. The propylene glycol extract of Bupleurum is dissolved in a water-propylene glycol mixture to obtain a propylene glycol extract liquid of Bupleurum. A volume ratio of the water to the propylene glycol in the water-propylene glycol mixture is 10:1. According to a mass ratio of the dexborneol to the propylene glycol extract of Bupleurum of 1.5:1, the dexborneol liquid and the propylene glycol extract liquid of Bupleurum are prepared, and then mixed to thereby obtain the pain relief formulation. A concentration of the dexborneol in the pain relief formulation is 75 ng/mL.

### Embodiment 3

A pain relief formulation is composed of dexborneol, propylene glycol extract of Bupleurum, and a pharmaceutically acceptable excipient. A mass ratio of the dexborneol to the propylene glycol extract of Bupleurum is 2:1.

A preparation method for the propylene glycol extract of Bupleurum is specifically as follows. Bupleurum is ground into Bupleurum powder with a particle size of 40 meshes. The Bupleurum powder is mixed with propylene glycol according to a ratio of 1 kg:8 L, then ultrasonic extracted for 30 min under a power of 400 W, and filtered with a 50-mesh screen, to thereby obtain a filtrate. The filtrate is collected and then freeze-dried at −20° C. to thereby obtain the propylene glycol extract of Bupleurum.

The pharmaceutically acceptable excipient is a combination of the propylene glycol and water.

A preparation method for the pain relief formulation is specifically as follows. The dexborneol is dissolved in the propylene glycol to obtain a dexborneol liquid. The propylene glycol extract of Bupleurum is dissolved in a water-propylene glycol mixture to obtain a propylene glycol extract liquid of Bupleurum. A volume ratio of the water to the propylene glycol in the water-propylene glycol mixture is 10:1. According to a mass ratio of the dexborneol to the propylene glycol extract of Bupleurum of 2:1, the dexborneol liquid and the propylene glycol extract liquid of Bupleurum are prepared, and then mixed to thereby obtain the pain relief formulation. A concentration of the dexborneol in the pain relief formulation is 75 ng/mL.

### Embodiment 4

A pain relief formulation is composed of dexborneol, propylene glycol extract of Bupleurum, and a pharmaceutically acceptable excipient. A mass ratio of the dexborneol to the propylene glycol extract of Bupleurum is 2.5:1.

A preparation method for the propylene glycol extract of Bupleurum is specifically as follows. Bupleurum is ground into Bupleurum powder with a particle size of 40 meshes. The Bupleurum powder is mixed with propylene glycol according to a ratio of 1 kg:8 L, then ultrasonic extracted for 30 min under a power of 400 W, and filtered with a 50-mesh screen, to thereby obtain a filtrate. The filtrate is collected and then freeze-dried at −20° C. to thereby obtain the propylene glycol extract of Bupleurum.

The pharmaceutically acceptable excipient is a combination of the propylene glycol and water.

A preparation method for the pain relief formulation is specifically as follows. The dexborneol is dissolved in the propylene glycol to obtain a dexborneol liquid. The propylene glycol extract of Bupleurum is dissolved in a water-propylene glycol mixture to obtain a propylene glycol extract liquid of Bupleurum. A volume ratio of the water to the propylene glycol in the water-propylene glycol mixture is 10:1. According to a mass ratio of the dexborneol to the propylene glycol extract of Bupleurum of 2.5:1, the dexborneol liquid and the propylene glycol extract liquid of Bupleurum are prepared, and then mixed to thereby obtain the pain relief formulation. A concentration of the dexborneol in the pain relief formulation is 75 ng/mL.

### Embodiment 5

A pain relief formulation is composed of dexborneol, propylene glycol extract of Bupleurum, and a pharmaceutically acceptable excipient. A mass ratio of the dexborneol to the propylene glycol extract of Bupleurum is 2:1.

A preparation method for the propylene glycol extract of Bupleurum is specifically as follows. Bupleurum is ground into Bupleurum powder with a particle size of 40 meshes. The Bupleurum powder is mixed with propylene glycol according to a ratio of 1 kg:8 L, then ultrasonic extracted for 30 min under a power of 400 W, and filtered with a 50-mesh screen, to thereby obtain a filtrate. The filtrate is collected and then freeze-dried at −20° C. to thereby obtain the propylene glycol extract of Bupleurum.

The pharmaceutically acceptable excipient is a combination of the propylene glycol and water.

A preparation method for the pain relief formulation is specifically as follows. The dexborneol is dissolved in the propylene glycol to obtain a dexborneol liquid. The propylene glycol extract of Bupleurum is dissolved in a water-propylene glycol mixture to obtain a propylene glycol extract liquid of Bupleurum. A volume ratio of the water to the propylene glycol in the water-propylene glycol mixture is 10:1. According to a mass ratio of the dexborneol to the propylene glycol extract of Bupleurum of 2:1, the dexborneol liquid and the propylene glycol extract liquid of Bupleurum are prepared, and then mixed to thereby obtain the pain relief formulation. A concentration of the dexborneol in the pain relief formulation is 60 ng/mL.

### Embodiment 6

A pain relief formulation is composed of dexborneol, propylene glycol extract of Bupleurum, and a pharmaceutically acceptable excipient. A mass ratio of the dexborneol to the propylene glycol extract of Bupleurum is 2:1.

A preparation method for the propylene glycol extract of Bupleurum is specifically as follows. Bupleurum is ground into Bupleurum powder with a particle size of 40 meshes. The Bupleurum powder is mixed with propylene glycol according to a ratio of 1 kg:8 L, then ultrasonic extracted for 30 min under a power of 400 W, and filtered with a 50-mesh screen, to thereby obtain a filtrate. The filtrate is collected and then freeze-dried at −20° C. to thereby obtain the propylene glycol extract of Bupleurum.

The pharmaceutically acceptable excipient is a combination of the propylene glycol and water.

A preparation method for the pain relief formulation is specifically as follows. The dexborneol is dissolved in the propylene glycol to obtain a dexborneol liquid. The propylene glycol extract of Bupleurum is dissolved in a water-propylene glycol mixture to obtain a propylene glycol extract liquid of Bupleurum. A volume ratio of the water to the propylene glycol in the water-propylene glycol mixture is 10:1. According to a mass ratio of the dexborneol to the propylene glycol extract of Bupleurum of 2:1, the dexborneol liquid and the propylene glycol extract liquid of Bupleurum are prepared, and then mixed to thereby obtain the pain relief formulation. A concentration of the dexborneol in the pain relief formulation is 80 ng/mL.

Differences among embodiment 1 through embodiment 4 lie in the mass ratio of the dexborneol to the propylene glycol extract of Bupleurum. In embodiment 1, the mass ratio of the dexborneol to the propylene glycol extract of Bupleurum is 1:1. In embodiment 2, the mass ratio of the dexborneol to the propylene glycol extract of Bupleurum is 1.5:1. In embodiment 3, the mass ratio of the dexborneol to the propylene glycol extract of Bupleurum is 2:1. In embodiment 4, the mass ratio of the dexborneol to the propylene glycol extract of Bupleurum is 2.5:1. Differences among embodiment 3, embodiment 5, and embodiment 6 lie in the concentration of the dexborneol in the pain relief formulation, which are respectively 75 ng/mL, 60 ng/mL, and 80 ng/mL.

Dosage forms of pain relief formulations prepared in embodiment 1 through embodiment 6 are all intramuscular injection, and the pain relief formulations prepared in embodiment 1 through embodiment 6 are all used through intramuscular injection.

Analgesic effects of the pain relief formulations prepared in embodiment 1 through embodiment 6 are discussed as follows.

### Experiment 1: Intramuscular Injection Experiment on Virus Models

### (1) Main Experiment Materials and Animals

African green monkey kidney (CV-1) cells are purchased from Shanghai Yaji Biotechnology Co., Ltd., with a brand of Yaji Biotechnology™.

A complete medium is Dulbecco's modified eagle medium/nutrient mixture F-12 (DMEM/F12).

Weights of female mice are in a range of 29.8 g to 30.2 g.

### (2) Grouping and Modeling

The CV-1 cells are suspended in the complete medium to obtain a cell suspension. The cell suspension is inoculated onto a culture dish at an inoculation concentration of 5×10⁵ cells per square centimeter. CV-1 cells in the cell suspension inoculated onto the culture dish are infected with varicella-zoster virus. When more than 80% of the CV-1 cells on the culture dish are infected with the varicella-zoster virus, CV-1 cells infected with the varicella-zoster virus are collected, then washed with a phosphate-buffered saline with a concentration of 0.1 moles per liter (mol/L) and a potential of hydrogen (pH) of 7.2, and resuspended to obtain a viral inoculum with a concentration of 10×10⁷ cells per 100 microliters (μL). Specifically, the varicella-zoster virus is varicella-zoster virus, reduced-Oka (VZV-rOka), provided by Xiamen University.

132 female mice are randomly divided into 11 groups, with 12 female mice in each group. Names of the 11 groups are respectively blank group, model group, dexborneol group, propylene glycol extract of Bupleurum group, embodiment 1 group, embodiment 2 group, embodiment 3 group, embodiment 4 group, embodiment 5 group, embodiment 6 group, and medicine control group.

Each of female mice in the blank group is intramuscularly injected with 50 μL of normal saline at a left plantar region.

Each of female mice in the other groups is intramuscularly injected with 50 μL of the viral inoculum at the left plantar region. From a first day after intramuscular injection, changes in paw withdrawal thresholds of female mice in experimental groups (i.e., the other groups) are observed. The paw withdrawal thresholds are measured by using a pressure analgesia meter. The paw withdrawal thresholds of the female mice begin to continuously decrease from a second day after the intramuscular injection of the virus inoculum, and on a twelfth day after the intramuscular injection of the virus inoculum, the paw withdrawal thresholds drop to a lower level and stabilize at certain pain threshold levels, indicating that postherpetic neuralgia models are successfully established.

### (3) Treatment

As a healthy control group, each of the female mice in the blank group is intramuscularly injected with 0.9 grams per 100 milliliters (g/100 mL) normal saline.

Each of female mice in the model group is intramuscularly injected with 0.9 g/100 mL normal saline.

Each of female mice in the dexborneol group is intramuscularly injected with 75 ng/mL propylene glycol solution of dexborneol to observe pain-relieving effect of the dexborneol alone in the female mice.

Each of female mice in the propylene glycol extract of Bupleurum group is intramuscularly injected with 75 ng/mL propylene glycol extract liquid of Bupleurum to observe pain-relieving effect of the propylene glycol extract of Bupleurum alone in the female mice.

Each of female mice in the embodiment 1 group is intramuscularly injected with the pain relief formulation prepared in embodiment 1.

Each of female mice in the embodiment 2 group is intramuscularly injected with the pain relief formulation prepared in embodiment 2.

Each of female mice in the embodiment 3 group is intramuscularly injected with the pain relief formulation prepared in embodiment 3.

Each of female mice in the embodiment 4 group is intramuscularly injected with the pain relief formulation prepared in embodiment 4.

Each of female mice in the embodiment 5 group is intramuscularly injected with the pain relief formulation prepared in embodiment 5.

Each of female mice in the embodiment 6 group is intramuscularly injected with the pain relief formulation prepared in embodiment 6.

Each of female mice in the medicine control group is intramuscularly injected with a pain relief formulation of the medicine control group.

Specific intramuscular injection volumes are shown in table 1.

During the intramuscular injection experiment on the virus models, the propylene glycol solution of dexborneol is obtained by dissolving the dexborneol in the propylene glycol. The propylene glycol extract liquid of Bupleurum is obtained by dissolving the propylene glycol extract of Bupleurum in the water-propylene glycol mixture, and in the water-propylene glycol mixture, the volume ratio of the water to the propylene glycol is 10:1. A preparation process of the pain relief formulation of the medicine control group is similar as that of the pain relief formulation prepared in embodiment 3, but during the preparation process of the pain relief formulation of the medicine control group, the propylene glycol extract of Bupleurum is replaced by a water extract of Bupleurum. A preparation method for the water extract of Bupleurum is specifically as follows. The Bupleurum is ground into the Bupleurum powder with the particle size of 40 meshes. The Bupleurum powder is mixed with deionized water according to a ratio of 1 kg:8 L, then ultrasonic extracted for 30 min under the power of 400 W, and filtered with the 50-mesh screen, to thereby obtain a water-extracted filtrate. The water-extracted filtrate is collected and then freeze-dried at −20° C. to thereby obtain the water extract of Bupleurum.

TABLE 1
Treatment methods for female mice in different groups

| | | Intramuscular |
|---|---|---|
| | | (milliliter, |
| Group name | Substance for intramuscular injection | abbreviated as mL) |
| Blank group | 0.9 g/100 mL normal saline | 0.3 |
| Model group | 0.9 g/100 mL normal saline | 0.3 |
| Dexborneol group | 75 ng/mL propylene glycol solution of | 0.3 |
| | dexborneol | |
| Propylene glycol extract of | 75 ng/mL propylene glycol extract | 0.3 |
| Bupleurum group | liquid of Bupleurum | |
| Embodiment 1 group | the pain relief formulation prepared in | 0.3 |
| | embodiment 1 with the concentration of | |
| | the dexborneol of 75 ng/mL and a | |
| | concentration of the propylene glycol | |
| | extract of Bupleurum of 75 ng/mL | |
| Embodiment 2 group | the pain relief formulation prepared in | 0.3 |
| | embodiment 2 with the concentration of | |
| | the dexborneol of 75 ng/mL and the | |
| | concentration of the propylene glycol | |
| | extract of Bupleurum of 50 ng/mL | |
| Embodiment 3 group | the pain relief formulation prepared in | 0.3 |
| | embodiment 3 with the concentration of | |
| | the dexborneol of 75 ng/mL and the | |
| | concentration of the propylene glycol | |
| | extract of Bupleurum of 37.5 ng/mL | |
| Embodiment 4 group | the pain relief formulation prepared in | 0.3 |
| | embodiment 4 with the concentration of | |
| | the dexborneol of 75 ng/mL and the | |
| | concentration of the propylene glycol | |
| | extract of Bupleurum of 30 ng/mL | |
| Embodiment 5 group | the pain relief formulation prepared in | 0.3 |
| | embodiment 5 with the concentration of | |
| | the dexborneol of 60 ng/mL and the | |
| | concentration of the propylene glycol | |
| | extract of Bupleurum of 30 ng/mL | |
| Embodiment 6 group | the pain relief formulation prepared in | 0.3 |
| | embodiment 6 with the concentration of | |
| | the dexborneol of 80 ng/mL and the | |
| | concentration of the propylene glycol | |
| | extract of Bupleurum of 40 ng/mL | |
| Medicine control group | the pain relief formulation of the | |
| | medicine control group with the | |
| | concentration of the dexborneol of 60 | 0.3 |
| | ng/mL and the concentration of the | |
| | water extract of Bupleurum of 30 ng/mL | |

### (4) Pain Index Test

Mechanical paw withdrawal threshold of right hind limb of each of the female mice is measured by using a Von Frey Filament tactile analgesia meter. Stimulus intensities are recorded when the female mouse exhibits behaviors such as paw lifting, licking, or avoidance. Each of the female mice is tested five times, and an average value of remaining three records of the stimulus intensities after removing maximum and minimum values of the stimulus intensities is taken as the mechanical paw withdrawal threshold. Testing results are shown in table 2.

TABLE 2
Treatment results of the female mice in different groups

| | Mechanical paw |
|---|---|
| Group name | withdrawal threshold (g) |
| Blank group | 26.8 ± 0.5 |
| Model group | 5.3 ± 0.6 |
| Dexborneol group | 13.8 ± 0.6 |
| Propylene glycol extract of Bupleurum group | 10.7 ± 0.8 |
| Embodiment 1 group | 20.4 ± 0.5 |
| Embodiment 2 group | 22.0 ± 0.1 |
| Embodiment 3 group | 25.9 ± 0.2 |
| Embodiment 4 group | 21.3 ± 0.3 |
| Embodiment 5 group | 22.9 ± 0.2 |
| Embodiment 6 group | 23.8 ± 0.3 |
| Medicine control group | 16.6 ± 0.2 |

Herpes zoster is an infectious disease affecting nerves and skin, which is caused by the varicella-zoster virus. The herpes zoster often occurs in a trunk, including a chest, an abdomen and a back, which mainly causes pain and rash without other special symptoms, and generally has no special sequelae after timely treatment. Some patients with the herpes zoster may suffer from chronic pain such as the postherpetic neuralgia. Patients with postherpetic neuralgia usually experience long-term pain, which mainly includes neuralgia, stabbing pain, numbness, etc. Nature of the long-term pain is diverse, and can be burn-like, electric shock-like, knife-like, needle-like, or tear-like. The postherpetic neuralgia affects normal life of patients, therefore, it is necessary to develop formulations to relieve the postherpetic neuralgia.

It can be seen from the treatment results shown in table 2, the pain relief formulations in embodiment 1 group through embodiment 6 group can significantly relieve the postherpetic neuralgia. Effect in relieving the postherpetic neuralgia of the medicine control group, the dexborneol group, and the propylene glycol extract of Bupleurum group is significantly lower than that of the embodiment 1 group through embodiment 6 group, P<0.05.

### Experiment 2: Intramuscular Injection Experiment on Acute Pain Models

### (1) Experiment Animals

Sprague-Dawley (SD) rats have weights in a range of 239.8 g to 260.2 g.

### (2) Modeling

132 SD rats are randomly divided into 11 groups, with 12 SD rats in each group. Names of the 11 groups are respectively blank group, model group, dexborneol group, propylene glycol extract of Bupleurum group, embodiment 1 group, embodiment 2 group, embodiment 3 group, embodiment 4 group, embodiment 5 group, embodiment 6 group, and medicine control group.

The SD rats are fasted in six hours before modeling and deprived of water in one hour before modeling. The SD rats are placed in a 1 liter (L) transparent glass container soaked with isoflurane liquid cotton balls to make the SD rats lose consciousness. A 1-centimeter (cm) longitudinal incision is made on a plantar surface of a hind paw of each of the SD rats, starting 0.5 cm proximal to a toe base and extending toward digits. Skin of the SD rat is incised, and underlying plantar muscle of the SD rat is gently elevated using ophthalmic forceps and longitudinally incised while preserving its origin, insertion, and attachments. After pressing to stop bleeding, the skin is sutured with a fine suture to complete establishment of the acute pain models.

### (3) Treatment

As a healthy control group, each of SD rats in the blank group is intramuscularly injected with 0.9 g/100 mL normal saline.

Each of SD rats in the model group is intramuscularly injected with 0.9 g/100 mL normal saline.

Each of SD rats in the dexborneol group is intramuscularly injected with 75 ng/mL propylene glycol solution of dexborneol to observe pain-relieving effect of the dexborneol alone in the SD rats.

Each of SD rats in the propylene glycol extract of Bupleurum group is intramuscularly injected with 75 ng/mL propylene glycol extract liquid of Bupleurum.

Each of SD rats in the embodiment 1 group is intramuscularly injected with the pain relief formulation prepared in embodiment 1.

Each of SD rats in the embodiment 2 group is intramuscularly injected with the pain relief formulation prepared in embodiment 2.

Each of SD rats in the embodiment 3 group is intramuscularly injected with the pain relief formulation prepared in embodiment 3.

Each of SD rats in the embodiment 4 group is intramuscularly injected with the pain relief formulation prepared in embodiment 4.

Each of SD rats in the embodiment 5 group is intramuscularly injected with the pain relief formulation prepared in embodiment 5.

Each of SD rats in the embodiment 6 group is intramuscularly injected with the pain relief formulation prepared in embodiment 6.

Each of SD rats in the medicine control group is intramuscularly injected with the pain relief formulation of the medicine control group.

During the intramuscular injection experiment on the acute pain models, the propylene glycol solution of dexborneol is obtained by dissolving the dexborneol in the propylene glycol. The propylene glycol extract liquid of Bupleurum is obtained by dissolving the propylene glycol extract of Bupleurum in the water-propylene glycol mixture, and in the water-propylene glycol mixture, the volume ratio of the water to the propylene glycol is 10:1. The preparation process of the pain relief formulation of the medicine control group is similar as that of the pain relief formulation prepared in embodiment 3, but during the preparation process of the pain relief formulation of the medicine control group, the propylene glycol extract of Bupleurum is replaced by the water extract of Bupleurum. The preparation method for the water extract of Bupleurum is specifically as follows. The Bupleurum is ground into the Bupleurum powder with the particle size of 40 meshes. The Bupleurum powder is mixed with the deionized water according to the ratio of 1 kg:8 L, then ultrasonic extracted for 30 min under the power of 400 W, and filtered with the 50-mesh screen, to thereby obtain the water-extracted filtrate. The water-extracted filtrate is collected and then freeze-dried at −20° C. to thereby obtain the water extract of Bupleurum.

Specific intramuscular injection volumes are shown in table 3.

TABLE 3
Treatment methods for SD rats in different groups

| | | Intramuscular injection |
|---|---|---|
| Group name | Substance for intramuscular injection | volume (mL) |
| Blank group | 0.9 g/100 mL normal saline | 1 |
| Model group | 0.9 g/100 mL normal saline | 1 |
| Dexborneol group | 75 ng/mL propylene glycol solution of | 1 |
| | dexborneol | |
| Propylene glycol extract of | 75 ng/mL propylene glycol extract liquid | 1 |
| Bupleurum group | of Bupleurum | |
| Embodiment 1 group | the pain relief formulation prepared in | 1 |
| | embodiment 1 with the concentration of | |
| | the dexborneol of 75 ng/mL and the | |
| | concentration of the propylene glycol | |
| | extract of Bupleurum of 75 ng/mL | |
| Embodiment 2 group | the pain relief formulation prepared in | 1 |
| | embodiment 2 with the concentration of | |
| | the dexborneol of 75 ng/mL and the | |
| | concentration of the propylene glycol | |
| | extract of Bupleurum of 50 ng/mL | |
| Embodiment 3 group | the pain relief formulation prepared in | 1 |
| | embodiment 3 with the concentration of | |
| | the dexborneol of 75 ng/mL and the | |
| | concentration of the propylene glycol | |
| | extract of Bupleurum of 37.5 ng/mL | |
| Embodiment 4 group | the pain relief formulation prepared in | 1 |
| | embodiment 4 with the concentration of | |
| | the dexborneol of 75 ng/mL and the | |
| | concentration of the propylene glycol | |
| | extract of Bupleurum of 30 ng/mL | |
| Embodiment 5 group | the pain relief formulation prepared in | 1 |
| | embodiment 5 with the concentration of | |
| | the dexborneol of 60 ng/mL and the | |
| | concentration of the propylene glycol | |
| | extract of Bupleurum of 30 ng/mL | |
| Embodiment 6 group | the pain relief formulation prepared in | 1 |
| | embodiment 6 with the concentration of | |
| | the dexborneol of 80 ng/mL and the | |
| | concentration of the propylene glycol | |
| | extract of Bupleurum of 40 ng/mL | |
| Medicine control group | the pain relief formulation of the | 1 |
| | medicine control group with the | |
| | concentration of the dexborneol of 60 | |
| | ng/mL and the concentration of the | |
| | water extract of Bupleurum of 30 ng/mL | |

### (4) Pain Index Test

Mechanical paw withdrawal thresholds of the SD rats in different groups are tested according to the testing method in experiment 1, and testing results are shown in table 4.

TABLE 4
Treatment results of the SD rats in different groups

| | Mechanical paw |
|---|---|
| Group name | withdrawal threshold (g) |
| Blank group | 38.1 ± 0.6 |
| Model group | 11.6 ± 0.5 |
| Dexborneol group | 20.1 ± 0.1 |
| Propylene glycol extract of Bupleurum group | 16.0 ± 0.4 |
| Embodiment 1 group | 31.6 ± 0.5 |
| Embodiment 2 group | 33.9 ± 0.3 |
| Embodiment 3 group | 37.1 ± 0.2 |
| Embodiment 4 group | 33.5 ± 0.2 |
| Embodiment 5 group | 35.0 ± 0.1 |
| Embodiment 6 group | 35.4 ± 0.3 |
| Medicine control group | 21.3 ± 0.1 |

Relieving the acute pain has significant implications in various aspects, including physiology, psychology, disease treatment, and social economy. Relieving the acute pain can prevent physiological disorders, promote wound healing, alleviate anxiety and fear, improve sleep quality, enhance patient compliance, and reduce occupation of medical resources.

It can be seen from the treatment results shown in table 4, the pain relief formulations in embodiment 1 group through embodiment 6 group can significantly relieve the acute pain. Effect in relieving the acute pain of the medicine control group, the dexborneol group, and the propylene glycol extract of Bupleurum group are significantly lower than that of the embodiment 1 group through embodiment 6 group, P<0.05.

It should be noted that, when numerical ranges are involved in the disclosure, it should be understood that two endpoints of each numerical range and any numerical value between the two endpoints can be selected. Because steps and methods used are the same as those of the embodiments, part of embodiments of the disclosure are described to avoid redundancy. Although the part of embodiments of the disclosure have been described, those skilled in the art can make additional changes and modifications to these embodiments once they know a creative concept of the disclosure, and these changes and modifications shall fall within a scope of protection of the disclosure.

Apparently, those skilled in the art can make various modifications and variations to the disclosure without departing from a spirit and the scope of the disclosure. When these modifications and variations made to the disclosure are within a scope of equivalent technology of the disclosure, the disclosure is also intended to include these modifications and variations.

What is claimed is:

1. An application of a pain relief formulation for preparing a medicine for relieving postherpetic neuralgia, wherein the pain relief formulation comprises dexborneol, a propylene glycol extract of Bupleurum, and a pharmaceutically acceptable excipient;
wherein a mass ratio of the dexborneol to the propylene glycol extract of Bupleurum is (1-2.5):1; and
wherein the propylene glycol extract of Bupleurum is prepared through the following method:
grinding Bupleurum into Bupleurum powder, mixing the Bupleurum powder with propylene glycol, followed by ultrasonic extracting and filtering to obtain a filtrate, collecting the filtrate, and drying the filtrate to obtain the propylene glycol extract of the Bupleurum.

2. The application of the pain relief formulation for preparing the medicine for relieving the postherpetic neuralgia as claimed in claim 1, wherein the mass ratio of the dexborneol to the propylene glycol extract of Bupleurum is 2:1.

3. The application of the pain relief formulation for preparing the medicine for relieving the postherpetic neuralgia as claimed in claim 1, wherein conditions for the ultrasonic extracting comprise a power of 400 watts (W) and a duration of 30 minutes (min).

4. The application of the pain relief formulation for preparing the medicine for relieving the postherpetic neuralgia as claimed in claim 3, wherein the pharmaceutically acceptable excipient is:
a combination of the propylene glycol and water,
the propylene glycol, or
a combination of hydroxypropyl-β-cyclodextrin, the propylene glycol, and the water.

5. The application of the pain relief formulation for preparing the medicine for relieving the postherpetic neuralgia as claimed in claim 1, wherein a concentration of the dexborneol in the pain relief formulation is in a range of 60 nanograms per milliliter (ng/mL) to 80 ng/mL.

6. The application of the pain relief formulation for preparing the medicine for relieving the postherpetic neuralgia as claimed in claim 1, wherein a concentration of the dexborneol in the pain relief formulation is 75 ng/mL.

7. The application of the pain relief formulation for preparing the medicine for relieving the postherpetic neuralgia as claimed in claim 1, wherein a preparation method for the pain relief formulation comprises the following steps:
mixing the dexborneol, the propylene glycol extract of Bupleurum, and the pharmaceutically acceptable excipient to obtain a mixture, wherein the mixture is the pain relief formulation.

8. The application of the pain relief formulation for preparing the medicine for relieving the postherpetic neuralgia as claimed in claim 4, wherein a preparation method for the pain relief formulation comprises the following steps:
dissolving the dexborneol in the propylene glycol to obtain dexborneol liquid;
dissolving the propylene glycol extract of Bupleurum in a water-propylene glycol mixture to obtain a propylene glycol extract liquid of Bupleurum; and
mixing the dexborneol liquid with the propylene glycol extract liquid of Bupleurum to thereby obtain the pain relief formulation.

9. The application of the pain relief formulation for preparing the medicine for relieving the postherpetic neuralgia as claimed in claim 1, wherein the medicine for relieving the postherpetic neuralgia is a medicine for relieving pain caused by varicella-zoster virus.
